(19) United States
(12) Patent Application Publication
(10) Pub. No.: US 2007/0238895 A1
(43) Pub. Date: Oct. 11, 2007

(54) N-protected aminoindanes and methods of their preparation

(76) Inventors: Lee Boulton, Royston (GB); Celine Praquin, Canterbury (GB)

Correspondence Address: KENYON & KENYON LLP, ONE BROADWAY, NEW YORK, NY, 10004, US

(21) Appl. No.: 11/594,499

(22) Filed: Nov. 7, 2006

Related U.S. Application Data
(60) Provisional application No. 60/737,093, filed on Nov. 15, 2005.
(60) Provisional application No. 60/734,626, filed on Nov. 7, 2005.

(51) Int. Cl. C07C 269/00

(52) U.S. Cl. 560/157

(57) ABSTRACT

The present invention relates to carbamoyl N-protected aminoindane derivatives, preferably carbamoyl N-Boc aminoindane derivatices, which are useful intermediates for the preparation of compounds used in the treatment of various CNS disorders, and their preparation.

### CROSS REFERENCE TO RELATED APPLICATIONS

The present application claims the benefit of the following U.S. Provisional Patent Application No. 60/734,626, filed Nov. 7, 2005, and No. 60/737,093, filed Nov. 15, 2005. The contents of this application is incorporated herein by reference.

### FIELD OF THE INVENTION

The present invention relates to carbamoyl N-protected aminoindane derivatives, which are useful intermediates for the preparation of compounds used in the treatment of various CNS disorders, and their preparation.

### BACKGROUND OF THE INVENTION

Carbamoyl N-protected aminoindane derivatives, preferably carbamoyl N-Boc aminoindane derivatives, are intermediates useful for preparing carbamoyl aminoindane derivatives such as for example Ladostigil. The chemical structure of a carbamoyl N-Boc aminoindane derivative is:

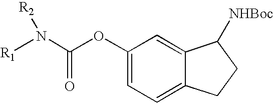

The carbamoyl aminoindane derivatives, which may be prepared from the intermediates prepared by the present invention, have been shown to be effective in Alzheimer's disease. In Alzheimer's type dementia a common pathological feature is the lack of the neurotransmitter acetylcholine. This has led to the development of acetylcholine esterase inhibitors for use in the treatment of Alzheimer's disease. Ladostigil is an example of such carbamoyl aminoindane derivative and is an active pharmaceutical ingredient which has shown to be effective in animal models of Alzheimer's disease. It also contains a (R)-N-propargyl aminoindane moiety which is a monoamine oxidase type B inhibitor. Ladostigil is disclosed in Weinstock, M. et al. J Neuronal Transm. (2000) [suppl]; 60: 157-169, Weinstock, M. et al. Development Research (2000); 50:216-222, Sterling J. et al. J. Med. Chem. 2002; 45:5260-5279, Weinstock M. et al. Psychopharmacology 2002;160:318-324; and Yogev-Falach et al. FASEB J. 2002; Oct. 16(12):1674-1676.

A method for preparing carbamoyl aminoindane derivatives is described in U.S. Pat. No. 6,303,650. The '650 patent describes the preparation of carbamoyl propargyl aminoindane derivatives by preparing a carbamoyl aminoindane from a hydroxy aminoindane and a carbamoylhalogenide. The carbamoyl aminoindan is reacted with an appropriate propargyl compound to prepare a carbamoyl propargyl aminoindane.

The methods of preparing pharmaceutically active carbamoyl aminoindane derivatives as described often results in racemic mixtures of the various enantiomers of the desired compounds. Typically, however, only one optically active enantiomer of the compound is pharmaceutically active. Therefore, there is a need to prepare optically active enantiomers of the carbamoyl aminoindane derivatives. Useful for the preparation of such compounds are stereospecific/enantiomeric intermediates for processes to prepare an enantiomer of a carbamoyl aminoindane derivative.

U.S. patent application Ser No. 11/______, filed Nov. 6, 2006, which claims priority from U.S. provisional applications Ser. No. 60/733,744 and 60/737,094, filed Nov. 4, 2005 and Nov. 15, 2005 respectively, which is incorporated herein in its entirety by reference, describes the asymmetric hydrogenation of carbamoyl acyl enamides to form carbamoyl acetamide indans. Preparation of useful intermediates for the synthesis of carbamoyl aminoindane derivatives further requires removal of the stable acetyl group from such carbamoyl acetamide indans without effecting the stereochemistry. Generally, the direct cleavage of the stable acetyl group from such carbamoyl acetamide indane requires harsh conditions which are not suitable for preserving stereochemistry and preparing the carbamoyl aminoindane in a high enantiomeric excess.

Burk et al. (J. Org. Chem; 1997; 62, 7054-7057) describe a two step process for converting a N-acetamido amino acid to a N-(tert butoxycarbonyl) amino acid (N-Boc amino acid) while preserving the stereochemistry of the amino acid.

The present invention is directed to the preparation of enantiomers of N-protected, preferably N-Boc, aminoindanes, intermediates for the preparation of aminoindane derivatives, from carbamoyl substituted amide indans, from which the amide carbonyl is cleaved while preserving the stereochemistry of the (N-protected) carbamoyl amino indane. The introduction of a protecting group, preferably a Boc group, in preparing such N-protected aminoindane while preserving the stereochemistry involves a complex reaction process. Consequently, various substitutions on the amide indane, may affect the process of introducing a protecting group differently, for example some substituents may result in a racemic mixture or even prevent the introduction of a protecting group on an enantiomer of such substituted amide indane. The present invention thus provides a process of preparing carbamoyl N-protected aminoindanes, preferably carbamoyl N-Boc protected aminoindanes, providing optically active carbamoyl N-protected aminoindanes as appropriate intermediates for the synthesis of aminoindane derivatives, such as Ladostigil.

### SUMMARY OF THE INVENTION

The present invention provides an isolated enantiomer of a carbamoyl N-protected aminoindane compound of formula I 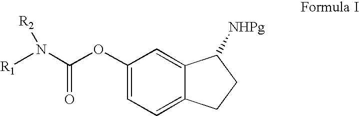
wherein R₁ and R₂ are each independently selected from a hydrogen, a straight, branched chain C₁₋₁₂ alkyl or benzyl group or p-OMe-phenyl and Pg is any protecting group which is easily cleaved under mild acidic conditions, preferably a Boc protecting group. These enantiomeric compounds are useful intermediates in preparing compounds used in the treatment of various CNS disorders. A preferred carbamoyl N-protected aminoindane, ethylmethylcarbamoyl N-Boc aminoindane, is useful as an intermediate in the synthesis of Ladostigil.

The present invention provides a method for preparing a carbamoyl N-protected, preferably N-Boc, aminoindane of formula I 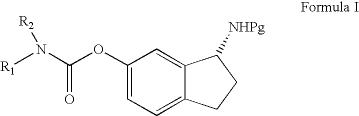
comprising the steps of

a) providing a carbamoyl amide indane of formula II, wherein the amide is preferably an acetamide,

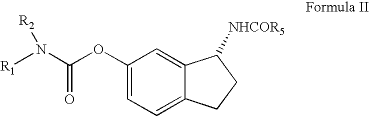

b) introducing a protecting group, preferably a Boc group, to the carbamoyl amide indane of formula II to form a carbamoyl N-protected amide indane of formula III, and

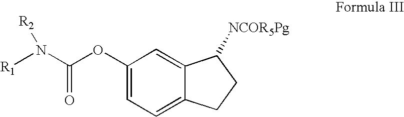

c) removing the amide carbonyl group from the carbamoyl N-protected amide indane of formula III to form the carbamoyl N-protected aminoindane of formula I, wherein R₁ and R₂ are each independently selected from hydrogen, a straight or branched chain C₁₋₁₂ alkyl, C₆₋₁₂ aryl, C₆₋₁₂ aralkyl or C₆₋₁₂ cycloalkyl or p-OMe-phenyl alkyl group, R₅ is methyl, ethyl or propyl, and Pg is any protecting group which is easily cleaved under mild acidic conditions, preferably Boc.

### DETAILED DESCRIPTION OF THE INVENTION

As used herein, the term;
DMAP refers to 4-dimethylaminopyridine;
Boc refers to a tert-butyloxycarbonyl protecting group;
Boc₂O refers to di-tert-butyl dicarbonate, or Boc anhydride;
THF refers to tetrahydrofuran;
DMF refers to dimethylformamide;
DCM refers to dichloromethane;
Carbamoyl refers to a radical/substituent group R₁R₂NCOO—; and
Indane refers to a compound having the following structure:

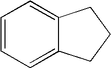

In one embodiment of the present invention there is provided a method for preparing a carbamoyl N-protected amide indane of formula III, preferably a carbamoyl N-BOC acetamide indane, 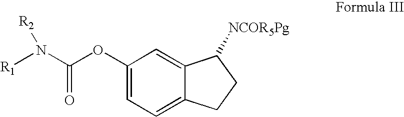
comprising the steps of

a) providing a carbamoyl amide indane of formula II, and

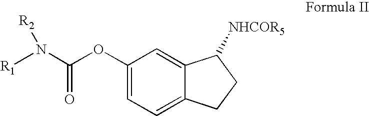

b) introducing a protecting group Pg to the carbamoyl amide indane of formula II to form a carbamoyl N-protected amide indane of formula III, wherein R₁ and R₂ are each independently selected from hydrogen, a straight or branched chain C₁₋₁₂ alkyl, C₆₋₁₂ aryl, C₆₋₁₂ aralkyl or C₆₋₁₂ cycloalkyl or p-OMe-phenyl alkyl group, R₅ is methyl, ethyl or propyl, and Pg is any protecting group which is easily cleaved under mild acidic condition. Preferably, R₁ is an ethyl group. Preferably, R₂ is a methyl group. Preferably, R₅ is methyl. Most preferably, R₁ is methyl and R₂ is ethyl and R₅ is methyl. Preferably, Pg is Boc.

The method of the present invention may further comprise preparing a carbamoyl N-protected aminoindane of formula I, by

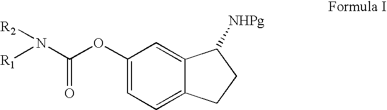

c) removing the amide carbonyl group from the carbamoyl N-protected amide indane of formula III, 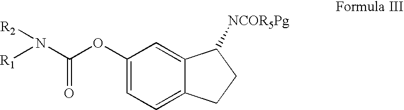
to form a carbamoyl N-protected aminoindane of formula I, wherein R₁ and R₂ are each independently selected from hydrogen, a straight or branched chain C₁₋₁₂ alkyl, C₆₋₁₂ aryl, C₆₋₁₂ aralkyl or C₆₋₁₂ cycloalkyl or p-OMe-phenyl alkyl group, R₅ is methyl, ethyl or propyl, and Pg is any protecting group which is easily cleaved under mild acidic conditions. Preferably, R₅ is methyl. Preferably, R₁ is an ethyl group and R₂ is a methyl group. Preferably, Pg is Boc.

A preferred embodiment of the method of the present invention provides a method for preparing a carbamoyl N-protected, preferably N-Boc, aminoindane of formula I 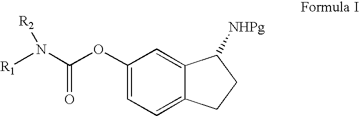
comprising the steps of

a) providing a carbamoyl amide indane of formula II, wherein the amide is preferably an acetamide,

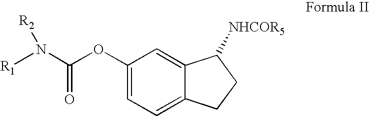

b) introducing a protecting group, preferably a Boc group, to the carbamoyl amide indane of formula II to form a carbamoyl N-protected amide indane of formula III, and

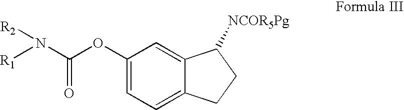

c) removing the amide carbonyl group from the carbamoyl N-protected amide indane of formula III to form the carbamoyl N-protected aminoindane of formula I, wherein R₁ and R₂ are each independently selected from hydrogen, a straight or branched chain C₁₋₁₂ alkyl, C₆₋₁₂ aryl, C₆₋₁₂ aralkyl or C₆₋₁₂ cycloalkyl or p-OMe-phenyl alkyl group, R₅ is methyl, ethyl or propyl, and Pg is any protecting group which is easily cleaved under mild acidic conditions, preferably Boc.

The N-protecting group, Pg, is any protecting group which is easily cleaved under mild acidic conditions. Preferably, the protecting group is selected from the group consisting of tert-butyloxycarbonyl (Boc), 2-Trimethylsilylethyl carbamate, 1,1-Dimethylpropyonyl carbamate, 1-methyl-1-(4-biphenylyl)ethyl carbamate, 1-Methylcyclobutyl carbamate, 1-Adamantanyl carbamate, Vinyl carbamate. More preferably, the protecting group is Boc.

A carbamoyl amide indane of formula II can be provided by preparing this compound with any method which yields the enantiomeric compound of formula II in high enantiomeric excess and sufficient purity to prepare an enantiomeric carbamoyl N-protected amide indane of formula III. A preferred method of preparing such carbamoyl amide indane is described in the U.S. patent application Ser. No. 11/______, filed Nov. 6, 2006, which claims priority from U.S. provisional applications Ser. No. 60/733,744 and 60/737,094, filed Nov. 4, 2005 and Nov. 15, 2005 respectively. This co-pending application is incorporated herein in its entirety by reference. In this co-pending application a carbamoyl acetamide indane, which is included in a carbamoyl amide indane of formula II, is prepared by a method comprising the steps of,

a) providing an acyl enamide of formula IV, and

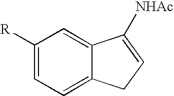

### Formula IV

b) hydrogenating the acyl enamide of formula IV in the presence of a catalyst to form the enantiomeric compound carbamoyl acetamide indane in an enantiomeric excess, wherein R is a carbamoyl represented by R₁R₂NCOO—, wherein R₁ and R₂ are each independently selected from hydrogen, a straight or branched chain C₁-C₆ alkyl group or a benzyl group. The catalysts are preferably asymmetic transition metal catalyst, in particular the homogeneous chiral ligand transition metal precatalyst of the formula [L-M X]Y, wherein L is a chiral ligand, M is a transition metal, X is an organic moiety and Y is an anion. A suitable catalyst for this reaction is selected from the group consisting of [(R,R)-Me-DuPhos Rh COD]BF₄, [(R,R)-Et-DuPhos Rh COD]BF₄, [(R,R)-Me-BPE Rh COD]BF₄, [(R,R)-Et-BPE Rh COD]BF₄, and [(S,S)-Ph-BPE Rh COD]BF₄.

Introduction of a protecting group, preferably a Boc group, to the carbamoyl amide indane of formula II (step b of the method of the present invention) may be carried out in a reaction mixture comprising the compound of formula II, a catalyst, preferably an organic catalyst, and a suitable protecting group reactant, for example, di-tert butyl dicarbonate (Boc₂O) for introducing a Boc protecting group, in an organic solvent. The reaction temperature for introducing the protecting group is maintained for a sufficient period to obtain the carbamoyl N-protected amide indane of formula III.

A preferred reaction temperature for introducing a protecting group to form a carbamoyl N-protected amide indane of formula III is from about 20° C. to about 55° C., more preferably from about 20° C. to about 40° C., most preferably about 40° C. The reaction period, the period of the maintaining this reaction temperature, is from about 15 hours to about 92 hours, preferably from about 18 hours to about 48 hours, more preferably from about 18 hours to about 24 hours. Alternatively, the reaction temperature is initially maintained at a temperature at about 15° C. to about 25° C., preferably at about 20° C., for a period of about 20 hours to about 24 hours and is subsequently increased to a temperature from about 40° C. to about 55° C. for a period of about 24 hours to about 72 hours. Preferably, the reaction temperature is in the alternative maintained at about 20° C. for about 24 hours and then increased to about 40° C. for an additional 24 hours.

A preferred reaction temperature for introducing a Boc group to form a carbamoyl N-Boc amide indane of formula III is from about 20° C. to about 55° C., more preferably from about 20° C. to about 40° C., most preferably about 40° C. The reaction period, the period of the maintaining this reaction temperature, is from about 15 hours to about 92 hours, preferably from about 18 hours to about 48 hours, more preferably from about 18 hours to about 24 hours. Alternatively, the reaction temperature is initially maintained at a temperature at about 15° C. to about 25° C., preferably at about 20° C., for a period of about 20 hours to about 24 hours and is subsequently increased to a temperature from about 40° C. to about 55° C. for a period of about 24 hours to about 72 hours. Preferably, the reaction temperature is in the alternative maintained at about 20° C. for about 24 hours and then increased to about 40° C. for an additional 24 hours.

Suitable catalysts to accelerate the introduction of a protecting group to form the carbamoyl N-protected amide indane of formula III may be selected from the group consisting of 4-dialkylaminopyridines, pyridine, and triethylamine. Preferably the organic catalyst is 4-dimethylaminopyridine (DMAP). The amount of catalyst present in the reaction mixture is preferably from about 0.20 molar equivalents to about 0.30 molar equivalents of the carbamoyl amide indane of formula II, preferably 0.25 molar equivalents of catalyst is present in the reaction mixture.

The suitable protecting group reactant is added to the reaction mixture in an amount from about 3.0 molar equivalents to about 6.0 molar equivalents of the carbamoyl amide indane of formula II. Preferably the amount of a suitable protecting group reactant in the reaction mixture for introducing a protecting group is from about 3.25 molar equivalents to about 5.0 molar equivalents, more preferably from about 3.5 molar equivalents to about 4.0 molar equivalents, most preferably about 3.5 molar equivalents.

The reactant di-tert butyl dicarbonate (Boc₂O) is added to the reaction mixture in an amount from about 3.0 molar equivalents to about 6.0 molar equivalents of the carbamoyl amide indane of formula II. Preferably the amount of Boc₂O in the reaction mixture for introducing a Boc group is from about 3.25 molar equivalents to about 5.0 molar equivalents, more preferably from about 3.5 molar equivalents to about 4.0 molar equivalents, most preferably about 3.5 molar equivalents.

Suitable solvents for the reaction mixture of introducing a protecting group, preferably the Boc protecting group, to the carbamoyl amide indane of formula II are selected from the group consisting of tetrahydrofuran (THF), dimethylformamide (DMF), dichloromethane (DCM), acetonitrile, and methanol. Preferred solvents include: tetrahydrofuran (THF), dimethylformamide (DMF) and dichloromethane (DCM), although THF is preferred.

In order to increase conversion of the carbamoyl amide indane of formula II to a carbamoyl N-protected amide indane of formula III the reaction mixture may further comprise an alkali metal alkoxide. Examples of alkali metal alkoxides include, but are not limited to, an alkali tert-butoxide, a metal methoxide or ethoxide. The preferable alkali tert-butoxide is potassium tert-butoxide while a preferable metal methoxide is sodium methoxide. Preferably, the amount of alkali metal tert-butoxide added to the reaction mixture is from about 0.75 molar equivalents to about 1.5 molar equivalents of the carbamoyl amide indane of formula II. More preferably, the amount of the alkali metal tert-butoxide is from about 1.0 molar equivalents to about 1.25 molar equivalents.

In order to increase conversion of the carbamoyl acetamide indane of formula II to a carbamoyl N-Boc amide indane of formula III, introducing a Boc group, the reaction mixture may further comprise an alkali metal alkoxide. Examples of alkali metal alkoxides include, but are not limited to, an alkali tert-butoxide, a metal methoxide or ethoxide. The preferable alkali tert-butoxide is potassium tert-butoxide while a preferable metal methoxide is sodium methoxide. Preferably, the amount of alkali metal tert-butoxide added to the reaction mixture is from about 0.75 molar equivalents to about 1.5 molar equivalents of the carbamoyl amide indane of formula II (e.g. carbamoyl acetamide indane of formula II). More preferably, the amount of the alkali metal tert-butoxide is from about 1.0 molar equivalents to about 1.25 molar equivalents.

The carbamoyl N-protected amide indane is subsequently converted to a carbamoyl N-protected aminoindane of formula I by removing the N-amide carbonyl group. Prior to removing the amide carbonyl group, the carbamoyl N-protected amide indane of formula III may be isolated from the reaction mixture and subsequently dissolved in a suitable organic solvent. Isolation of the carbamoyl N-protected amide indane of formula III may be carried out by any known suitable method, for example by evaporation of the organic solvent of the reaction mixture or through filtration with silica wherein the material is eluded from the silica with a suitable eluant, preferably a toluene/ethylacetate eluant, or a combination of such methods. Alternatively, the process of the present invention of introducing a protecting group and removing the amide carbonyl group to form a carbamoyl N-protected aminoindane of formula I may be carried out in a single reaction vessel without the need of isolating the carbamoyl N-protected amide indane compound of formula III. The process of the present invention is preferably carried out by isolating the carbamoyl N-protected amide indane of formula III and by subsequently dissolving this compound in a suitable organic solvent, prior to removing the amide carbonyl group.

The amide carbonyl group of the carbamoyl N-protected amide indane of formula III is preferably removed in step c) in a reaction mixture comprising the compound of formula III and a base in an organic solvent. The temperature of this reaction mixture is maintained for a period from about 2 hours to about 8 hours, preferably from about 4 hours to about 6 hours, more preferably for about 4 hours. The temperature is preferably from about 20° C. to about 40° C., more preferably about 25° C. Alternatively, this reaction can also be performed in a biphasic mixture of an aqueous solution of a base, preferably an aqueous solution of sodium hydroxide, and a organic solvent, such as toluene or ethyl acetate. When potassium carbonate is used in this reaction step, a non hindered alcohol is required.

The acetyl group of a carbamoyl N-Boc acetamide indane of formula III is preferably removed in step c) in a reaction mixture comprising the compound of formula III and a base in an organic solvent. The temperature of this reaction mixture is maintained for a period from about 2 hours to about 8 hours, preferably from about 4 hours to about 6 hours, more preferably for about 4 hours. The temperature is preferably from about 20° C. to about 40° C., more preferably about 25° C. Alternatively, this reaction can also be performed in a biphasic mixture of an aqueous solution of a base, preferably an aqueous solution of sodium hydroxide, and a organic solvent, such as toluene or ethyl acetate. When potassium carbonate is used in this reaction step, a non hindered alcohol is required.

Suitable organic solvents for the reaction mixture in removing the amide carbonyl group from the carbamoyl N-protected amide indane of formula III in step c) are C₁-C₄ alcohols. Preferably, the organic solvent is selected from the group consisting of methanol, ethanol, and isopropanol, more preferably the organic solvent is methanol. In a preferred single reaction vessel process of the invention an equal amount in volume of methanol is added to the reaction mixture of a carbamoyl N-protected amide indane of formula III produced in the previous step, step b).

Suitable bases for removing the amide carbonyl group in step c) are selected from the group consisting of alkali metal hydroxides, alkali metal carbonates, dialkylamines having the same or different C₁-C₄ alkyl groups, pyridine, morpholine, triethyl amine, and hydroxyl amine. Preferably, the alkali metal in these bases is sodium, lithium or potassium. More preferably, the base is selected from the group consisting of lithium hydroxide, sodium hydroxide and potassium carbonate, more preferably the base is potassium carbonate.

Further, the carbamoyl N-protected aminoindane of formula I as obtained in step c) may be isolated and/or purified. Isolation and/or purification of the carbamoyl N-protected aminoindane may be carried out by any known suitable method, for example crystallization, precipitation, concentration, evaporation, filtration or a combination thereof. Preferably, the method of the present invention further comprises the steps of

d) recovering crude carbamoyl N-protected aminoindane of formula I from the reaction mixture,

e) precipitating carbamoyl N-protected aminoindane of formula I from a solvent, and

f) recovering the precipitated carbamoyl N-protected aminoindane of formula I.

Preferably, recovering the crude carbamoyl N-protected aminoindane is carried out by extraction from the reaction mixture, preferably with an organic solvent, more preferably with ethylacetate, and subsequently filtering and concentrating the extract under reduced pressure. The solvent in which the carbamoyl N-protected aminoindane is precipitated is preferably an organic solvent, more preferably a C₁-C₄ alkyl alcohol, most preferably methanol. The precipitated carbamoyl N-protected aminoindane may be recovered by any known suitable method and is preferably recovered by filtration.

In another embodiment of the present invention there is provided an isolated enantiomer of a carbamoyl N-protected amide indane of formula III. Preferably, the isolated enantiomer is in an enantiomeric excess of at least 95%, more preferably at least 97%, most preferably at least 98%.

In another embodiment of the present invention there is provided an isolated enantiomer of a carbamoyl N-protected aminoindane of formula I. Preferably, the isolated enantiomer is in an enantiomeric excess of at least 95%, more preferably at least 97%, most preferably at least 98%. Such enantiomeric compounds are useful as intermediates in preparing compounds used in the treatment of various CNS disorders. In particular, these carbamoyl N-protected aminoindanes are useful as intermediates in preparing carbamoyl aminoindane derivatives of formula V: 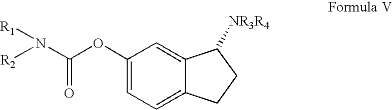
wherein R₃ and R₄ are independently a hydrogen, straight or branched C₁-C₄ alkyl, or a propargyl. A preferred compound of formula I, ethylmethylcarbamoyl N-Boc aminoindane, is useful as an intermediate in the synthesis of Lodastigil, an active pharmaceutical ingredient shown to be effective in animal models in the treatment of Alzheimer's disease.

Having described the invention with reference to certain preferred embodiments, other embodiments will become apparent to one skilled in the art from consideration of the specification. The disclosures of the prior art references referred to in this patent application are incorporated herein by reference. The invention is further defined by reference to the following examples describing in detail the process and compositions of the invention. It will be apparent to those skilled in the art that many modifications, both to materials and methods, may be practiced without departing from the scope of the invention.

### EXAMPLES

### Example 1

### Introduction of a Boc Group to a Carbamoyl Acetamide Indane

N,N-dimethylcarbamoyl acetamide indane was dissolved in an organic solvent in the presence of suitable reagents as shown in Table 1 to form a reaction mixture. The reaction mixture was maintained under the reaction conditions as shown in Table 1 for each of the various reaction mixtures. All reaction mixture converted the N,N-dimethylcarbamoyl acetamide indane to N,N-dimethylcarbamoyl N-Boc acetamide indane under these reaction conditions. The reaction mixture consisting of DMAP, Boc₂O, and KO^{t}Bu in THF resulted in the highest amount of conversion of at least 75% conversion.

TABLE 1
Reaction conditions in introducing N-Boc
to a dimethylcarbamoyl acetamide indane.

| Reagents (equiv.) | Solvent | Conditions |
|---|---|---|
| DMAP (0.25), Boc₂O (5.0) | THF | 20° C. for 20 hours then |
| | | 55° C. for 72 hours |
| DMAP (0.26), Boc₂O (3.3) | DMF | 20° C. for 24 hours then |
| | | 40° C. for 24 hours |
| DMAP (0.25), Boc₂O (3.25) | DCM | 20° C. for 24 hours then |
| | | 40° C. for 24 hours |
| DMAP (0.25), Boc₂O (3.6), | THF | 20° C. for 24 hours then |
| KO^{t}Bu (1.0) | | 40° C. for 24 hours |
| DMAP (0.25), Boc₂O (3.5), | THF | 40° C. for 24 hours |
| KO^{t}Bu (1.26) | | |

### Example 2

### Preparation of N-Boc-6-(N,N-dimethyl carbamate)-1-amino-indane

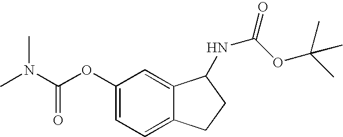 Potassium tert-butoxide (129 mg, 1.14 mmol) was added to a stirred solution of N-acetyl-6-(N,N-dimethyl carbamate)-1-amino-indane (276 mg, 1.05 mmol), DMAP (31 mg, 0.25 mmol) and Boc anhydride (894 mg, 4.09 mmol) in THF (10 mL). After stirring at 40° C. for 18 hours, TLC analysis indicated the reaction had gone to completion. The reaction mixture was concentrated under reduced pressure to give a brown oil. Ethyl acetate was added to the residue and the inorganics were filtered off. The liquors were concentrated under reduced pressure and the residue was purified by filtration through silica, using 33% toluene-ethyl acetate as eluant to give a yellow solid (295 mg). This material was dissolved in methanol (7 mL) and water (1 mL). Potassium carbonate (218 mg, 1.57 mmol) was added to the reaction mixture. After stirring 4 hours at room temperature, ¹H NMR analysis indicated complete conversion. Citric acid (2M, 4 mL) was added to the reaction mixture followed by water (4 mL) to attain pH 3. The product was extracted with ethyl acetate (2×10 mL). The combined organic extracts were washed with water (2×10 mL), dried (MgSO₄) and concentrated under reduced pressure to yield the desired compound as an orange solid (235 mg, 69%). ¹H NMR (400 MHz, d₆-DMSO) δ ppm 7.33 (1H, d, J5, NH), 7.24 (1H, d, J8, Ar), 6.95 (1H, dd, J8 and 2, Ar), 6.91 (1H, s, Ar), 5.03 (1H, dt, J8 and 8, CH), 3.08 (3H, s, MeN), 2.95 (3H, s, MeN), 2.92-2.88 (1H, m, CHH), 2.81-2.73 (1H, m, CHH), 2.45-2.37 (1H, m, CHH), 1.94-1.84 (1H, m, CHH) and 1.48 (9H, s, CMe₃).

### Example 3

### Preparation of N-Boc-6-(N,N-methyl-ethyl carbamate)-1-amino-indane

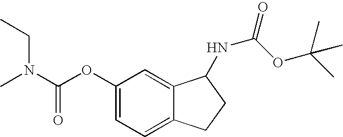 Potassium tert-butoxide (690 mg, 6.14 mmol) was added to a stirred solution of N-acetyl-6-(N,N-methyl-ethyl carbamate)-1-amino-indane (1.57 g, 5.68 mmol), DMAP (189 mg, 1.54 mmol) and Boc anhydride (5.22 g, 23.9 mmol) in THF (60 mL). After stirring at 40° C. for 19 hours, ¹H NMR analysis indicated the reaction was complete. The reaction mixture was cooled then poured into a saturated solution of ammonium chloride (100 mL) over 5 minutes. Water (20 mL) was added to the solution. The 2 layers were separated and the aqueous layer was further extracted with ethyl acetate (40 mL). The combined organic layers were washed with water (50 mL), dried (MgSO₄), filtered and concentrated under reduced pressure to give a brown oil. This was filtered through silica using 50% toluene-ethyl acetate as eluant to give a yellow liquid. The resulting residue was dissolved in methanol (100 mL). Potassium carbonate (1.46 g, 10.5 mmol) was added to the reaction mixture. After stirring 3.5 hours at room temperature, ¹H NMR analysis indicated complete conversion. Citric acid solution (2 N, 50 mL) was added to the reaction mixture to get pH 3. The solution was concentrated under reduced pressure to remove the methanol which was present. The product was extracted with ethyl acetate (2×40 mL). The combined organic extracts were washed with water (2×40 mL), dried (MgSO₄), filtered and concentrated under reduced pressure to yield the desired compound as a brown oil. Methanol (10 ml) was added to the residue and was removed again by concentration under reduced pressure to afford an orange solid. Heptane (15 mL) was added and the product was left to precipitate at room temperature. After stirring for 2 hours, the precipitate was collected by filtration to give the desired compound as a beige powder (1.00 g, 52%). ¹H NMR (400 MHz, d₆-DMSO) δ ppm 7.31 (1H, d, J8, NH), 7.22 (1H, d, J7, Ar), 6.93 (1H, dd, J8 and 2, Ar), 6.88 (1H, s, Ar), 5.00 (1H, dt, J8 and 8, CH), 3.44 (rotamer A, 1H, q, J7, CH₂), 3.36-3.32 (rotamer B, 1H, m, CH₂), 3.03 (rotamer A, 1.5H, s, CH₃N), 2.91 (rotamer B, 1.5H, s, CH₃N), 2.88-2.86 (1H, m, CHH), 2.79-2.70 (1H, m, CHH), 2.42-2.34 (1H, m, CHH), 1.91-1.81 (1H, m, CHH), 1.45 (9H, s, CMe₃), 1.20 (rotamer A, 1.5H, J6, Me) and 1.12 (rotamer B, 1.5H, J7, Me). Analysis of this material by chiral LC indicated it to be >98% e.e.

1. A method for preparing a carbamoyl N-protected aminoindane of formula I 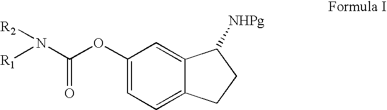
comprising the steps of
a) providing a carbamoyl amide indane of formula II, 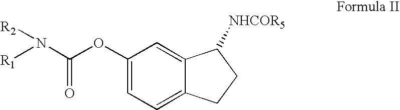
b) introducing a protecting group to the carbamoyl amide indane of formula II to form a carbamoyl N-protected amide indane of formula III, and 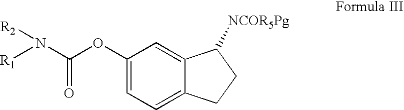
c) removing the amide carbonyl group from the carbamoyl N-protected amide indane of formula III to form a carbamoyl N-protected aminoindane of formula I, wherein R₁ and R₂ are each independently selected from a hydrogen, a straight, branched chain C₁₋₁₂ alkyl or benzyl group or p-OMe-phenyl, R₅ is a methyl, ethyl or propyl, and Pg is a protecting group which is easily cleaved under mild acidic conditions selected from Boc, 2-Trimethylsilylethyl carbamate, 1,1-Dimethylpropyonyl carbamate, 1-methyl-1-(4-biphenylyl)ethyl carbamate, 1-Methylcyclobutyl carbamate, 1-Adamantanyl carbamate, and Vinyl carbamate.

2. The method of claim 1, wherein R₅ is methyl and Pg is Boc.

3. The method of claim 1, wherein R₁ is ethyl.

4. The method of claim 1, wherein R₂ is methyl.

5. The method of claim 4, wherein R₁ is ethyl.

6. The method of claim 1, wherein introducing a protecting group (Pg) in step b) is carried out in a reaction mixture comprising the carbamoyl amide indane of formula II, a catalyst, and a protecting group reactant in an organic solvent.

7. The method of claim 6, wherein the protecting group (Pg) is Boc and the protecting group reactant is di-tert butyl dicarbonate.

8. The method of claim 6, wherein introducing a protecting group (Pg) in step b) is carried out a temperature of about 20° C. to about 55° C.

9. The method of claim 8, wherein the temperature is about 20° C. to about 40° C.

10. The method of claim 9, wherein the temperature is about 40° C.

11. The method of claim 8, wherein the temperature of the reaction mixture is maintained for a period of about 15 hours to about 92 hours.

12. The method of claim 11, wherein the period is from about 18 hours to about 48 hours.

13. The method of claim 12, wherein the period is from about 18 hours to about 24 hours.

14. The method of claim 6, wherein the reaction mixture is maintained for a period of about 20 hours to about 24 hours at a temperature of about 15° C. to about 25° C., and subsequently maintained for a period of about 24 hours to about 72 hours at a temperature of about 40° C. to about 55° C.

15. The method of claim 14, wherein the reaction mixture is maintained for a period of about 24 hours at a temperature of about 20° C., and subsequently maintained for a period of about 24 hours at a temperature of about 40° C.

16. The method of claim 6, wherein the catalyst is selected from the group consisting of 4-dialkylaminopyridines, and triethylamine.

17. The method of claim 16, wherein the catalyst is 4-dimethylaminopyridine (DMAP).

18. The method of claim 16, wherein the amount of catalyst in the reaction mixture is from about 0.20 molar equivalents to about 0.30 molar equivalents of the carbamoyl amide indane.

19. The method of claim 18, wherein the amount of catalyst in the reaction mixture is about 0.25 molar equivalents of the carbamoyl amide indane.

20. The method of claim 6, wherein the protecting group reactant is employed in an amount from about 3.0 molar equivalents to about 6.0 molar equivalents of the carbamoyl amide indane of formula II.

21. The method of claim 20, wherein the amount of protecting group reactant is from about 3.25 molar equivalents to about 5.0 molar equivalents.

22. The method of claim 21, wherein the amount of the protecting group reactant is about 3.5 molar equivalents.

23. The method of claim 6, wherein the organic solvent is selected from the group consisting of tetrahydrofuran (THF), dimethylformamide (DMF), dichloromethane (DCM), 1-methyl-2-pyrolidone (NMP), acetonitrile, pyridine, and methanol.

24. The method of claim 23, wherein the organic solvent is THF.

25. The method of claim 7, wherein the reactant di-tert butyl dicarbonate (Boc₂O) is employed in an amount from about 3.0 molar equivalents to about 6.0 molar equivalents of the carbamoyl amide indane of formula II.

26. The method of claim 25, wherein the amount of Boc₂O is from about 3.25 molar equivalents to about 5.0 molar equivalents.

27. The method of claim 26, wherein the amount of Boc₂O is about 3.5 molar equivalents.

28. The method of claim 25, wherein the reaction mixture further comprises an alkali metal tert-butoxide.

29. The method of claim 28, wherein the alkali metal tert-butoxide is potassium tert-butoxide.

30. The method of claim 29, wherein the amount of the alkali metal tert-butoxide in the reaction mixture is from about 0.75 molar equivalents to about 1.5 molar equivalents of the carbamoyl amide indane.

31. The method of claim 30, wherein the amount of the alkali tert-butoxide is from about 1.0 molar equivalents to about 1.25 molar equivalents.

32. The method of claim 6, wherein step b) further comprises isolating the carbamoyl N-protected amide indane of formula III.

33. The method of claim 6, wherein the method is carried out in a single reaction vessel.

34. The method of claim 1, wherein removing the amide carbonyl group in step c) is carried out in a reaction mixture comprising the carbamoyl N-protected amide indane of formula III and a base in an organic solvent.

35. The method of claim 34, wherein the reaction mixture is maintained at a reaction temperature of about 20° C. to about 40° C. for a period of about 2 hours to about 8 hours.

36. The method of claim 35, wherein the reaction temperature is about 25° C.

37. The method of claim 36, wherein the period is from about 4 hours to about 6 hours.

38. The method of claim 37, wherein the period is about 4 hours.

39. The method of claim 34, wherein the organic solvent is a C₁-C₄ alcohol.

40. The method of claim 39, wherein the organic solvent is selected from the group consisting of methanol, ethanol, and isopropanol.

41. The method of claim 40, wherein the organic solvent is methanol.

42. The method of claim 34, wherein the base is selected from the group consisting of alkali metal hydroxides, alkali metal carbonates, diamines, pyridine, morpholine, triethyl amine, and hydroxyl amine.

43. The method of claim 42, wherein the base is selected from the group consisting of lithium hydroxide, sodium hydroxide and potassium carbonate.

44. The method of claim 43, wherein the base is potassium carbonate.

45. The method of claim 44, wherein the solvent is a non-hindered alcohol.

46. The method of claim 34, wherein removing the amide carbonyl group in step c) is carried out in a biphasic mixture of a base in an aqueous solution and a organic solvent.

47. The method of claim 46, wherein the base is an aqueous solution of sodium hydroxide and the organic solvent is selected from the group containing toluene and ethyl acetate.

48. The method of claim 1, further comprising isolating the carbamoyl N-protected aminoindane of formula I as obtained in step c).

49. The method of claim 1, further comprising the steps of
d) recovering crude carbamoyl N-protected aminoindane of formula I from the reaction mixture,
e) precipitate carbamoyl N-protected aminoindane of formula I from a solvent, and
f) recovering the precipitated carbamoyl N-protected aminoindane of formula I.

50. The method of claim 49, wherein recovering the crude carbamoyl N-protected aminoindane is carried out by extraction from the reaction mixture and subsequently filtering and concentrating the extract under reduced pressure.

51. The method of claim 50, wherein the extraction is with an organic solvent.

52. The method of claim 51, wherein the solvent is ethyl acetate.

53. The method of claim 49, wherein the solvent in which the carbamoyl N-protected aminoindane is precipitated is preferably an organic solvent.

54. The method of claim 53, wherein the solvent is a C₁-C₄ alcohol.

55. The method of claim 54, wherein the C₁-C₄ alcohol is methanol.

56. The method of claim 49, wherein the precipitated carbamoyl N-protected aminoindane is recovered by filtration.

57. The method of claim 1, further comprising converting the carbamoyl N-protected aminoindane of formula I 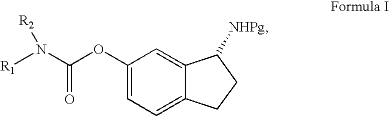
to an carbamoyl aminoindane of formula V 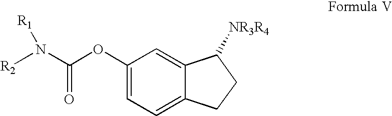
wherein R₃ and R₄ are independently a hydrogen, straight or branched C₁-C₄ alkyl, or propargyl.

58. The method of claim 57, wherein R₁ is ethyl, R₂ is methyl, R₃ is hydrogen, R₄ is propargyl, and Pg is Boc.

59. The method of claim 57, wherein the carbamoyl N-protected aminoindane of formula I is an isolated enantiomer.

60. An isolated enantiomer of a carbamoyl N-protected aminoindane of formula I 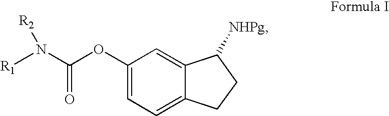
wherein R₁ and R₂ are each independently selected from a hydrogen, a straight, branched chain C₁₋₁₂ alkyl or benzyl group or p-OMe-phenyl, and Pg is a protecting group which is easily cleaved under mild acidic conditions selected from selected from Boc, 2-Trimethylsilylethyl carbamate, 1,1-Dimethylpropyonyl carbamate, 1-methyl-1-(4-biphenylyl)ethyl carbamate, 1-Methylcyclobutyl carbamate, 1-Adamantanyl carbamate, and Vinyl carbamate.

61. The isolated enantiomer of claim 60, wherein Pg is Boc.

62. The isolated enantiomer of claim 61, wherein R₁ is ethyl.

63. The isolated enantiomer of claim 61, wherein R₂ is methyl.

64. The isolated enantiomer of claim 63, wherein R₁ is ethyl.
